(19) United States
(12) Patent Application Publication
(10) Pub. No.: US 2005/0278009 A1
(43) Pub. Date: Dec. 15, 2005

(54) Electrode for electrostimulators

(76) Inventors: Gian Domenico Dalla Bona, Vicenza (IT); Joseph Oswaldo Diduro, Davenport, IA (US)

Correspondence Address: STURM & FIX LLP, 206 SIXTH AVENUE, SUITE 1213, DES MOINES, IA, 50309-4076, US

(21) Appl. No.: 11/077,508

(22) Filed: Mar. 10, 2005

(30) Foreign Application Priority Data
Mar. 10, 2004 (IT) VI2004A000046

(51) Int. Cl. A61N 1/04

(52) U.S. Cl. 607/115

(57) ABSTRACT

An electrode (1;31;51) for electrostimulators comprising an insulating housing (2;32;52) in which are located a first conductor element (3;33;53) projecting from the insulating housing (2;32;52); a second conductor element (4;34;54) also projecting from the insulating housing (3;32;52) and electrical conductor means (5;44) appropriate to connect the conductor elements (3, 4; 33,34; 53,54) to the electrostimulator. One of the conductor elements (3,4; 33, 34; 53, 54) is connected to a yielding structure (6; 37; 55) that ensures the simultaneous contact of both conductor elements (3, 4; 33, 34; 43, 54) with the human body during the therapeutic treatment.

The present invention relates to an electrode for electrostimulators, particularly adapted to be used for the obtaining of data useful for the diagnosis of painful pathologies and for the therapeutic treatment of the human body by means of the transmission of electric impulses to the skin.

It is known that electric stimulation is used in various fields of medicine and of physiotherapy in particular, in electrophysiology within experimental scope, in electrodiagnosis for the purpose of diagnoses and prognoses and, in particular, in electrotherapy for therapeutic purposes.

In this latter case, electrostimulation consists in subjecting a limited somatic area to the passage of an electric current of which the intensity, voltage, etc. are known.

Electrotherapy has the purpose of collecting different results depending on the type of electric current transmitted to the human body.

Thus, by way of example, using galvanic currents of low intensity, the electrotherapy facilitates the reabsorption of edemas besides exercising an analgesic action on the nervous system.

With galvanic currents of higher intensity, electrotherapy is used to causticize hemostases and coagulations as well as to dilate stenoses.

Electrotherapy applied for the treatment of peripheral paralyses uses alternate galvanic currents which, through the sudden variation of the electric current intensity, cause an excitant action of the muscle contractions in order to allow the checking of the threshold of the muscle movement.

Furthermore, electrotherapy can be used with low frequency currents which, through impulsive high-tension discharges on the muscle, allow the obtaining of beneficial effects on their atrophy.

Electrotherapy is applied using an ample variety of instruments or devices among which are known the electrodes that act on a circumscribed area of the human body.

In more details is known a bipolar electrode, subject of the International application WO91/19531, prevailingly used to check the threshold of the muscle movement.

Such an electrode is constituted by a round insulating body provided with a couple of conductor elements joined by electric current adduction means and an impulse generator.

The first conductor element is constituted by a peg that is substantially arranged in a central position and protruding from the part of the insulating body that is turned towards the area of the human body to be treated, while the second conductor element is constituted by a round cap also protruding from the insulating body on the side turned towards the area to be treated.

Both abovementioned conductor elements are secured to the insulating body from which they protrude at the same distance.

In order to generate the electric charge to treat or test the area of the body in question, they must be in simultaneous contact with the area itself.

Between the electrode and the mentioned area there is usually and advantageously inserted a gelatinous conductive substance or a synthetic polymer containing a conductive electrolyte salt to adapt the electrode to any feature of the skin and to ensure in this manner the continuity of the electric contact. This arrangement offers the further advantage to greatly soften the contact between the pegs of the electrode, many times of pointed shape, and the skin of the individual.

However, the electrode of the briefly described type and other similar ones present some disadvantages.

In particular, the principal disadvantage is due to the fact that the conductor elements constituting the electrode by protruding from the insulating body are solidly affixed to the electrode and therefore do not succeed in guaranteeing the passage of the current under all conditions.

As a matter of fact, this structural form of the electrode does not ensure that it adapts itself to the different shapes of the body of the individual of whom it shall show the pathology.

Therefore, the continuity of the contact of both poles of the electrode with the skin is not always ensured, with the obvious disadvantage of the lack of the electric charge transmission to the area to be treated.

Therefore, some areas of the human body with a specific shape cannot be treated or tested by electric stimulation with the electrode of described type.

To remedy such a disadvantage, conductive means of above-described type are inserted between the electrode and the area to be treated, the intensity of the electric current that arrives at the area to be treated is clearly lower which, in turns, reduces the effectiveness of the electro-stimulating treatment.

The object of the present invention is to overcome the mentioned disadvantages.

In particular, the principal object of the present invention is to obtain an electrode that ensures the continuous contact of both conductive poles of the electrode at any type of shape of the area of the human body subjected to the therapeutic or diagnostic treatment.

A further object is to obtain said contact without the need of having to insert additional conductive means of gelatinous or similar nature, thus increasing the effectiveness of the electro-stimulating treatment with respect to what the existing devices on the marketplace can offer.

Finally, a still further object is that of carrying out the therapeutic treatment without having to provoke large deformations or other lacerations to the individual's tissue being treated by means of an electrode.

The above enumerated objects are obtained by means of an electrode for electro-stimulation which, in accordance with the first claim, comprises an insulating body in which are installed:
at least one first conductor element protruding from said insulating body;
at least a second conductor element also protruding from said insulating body;
means of electric current conductors suitable to connect said conductor elements to the electrostimulator;
   which electrode is characterized by the fact that at least one of the conductor elements is connected to a flexible structure that ensures the simultaneous contact of both conductor elements with the human body during the therapeutic treatment.

The electrode in accordance with to the invention advantageously ensures the transmission of the electric impulses to the various shapes of the individual's body, guaranteeing the always active electric contact even without the recourse of inserting specific substances of gelatinous type.

Also advantageously, the electrode in accordance with to the invention produces excellent results, be they therapeutic or diagnostic, insofar as the originated electric current acts directly on the area to be treated and in a more effective manner compared with analogous devices of known type.

This allows to advantageously use the electrode in accordance with the invention not only to check the threshold of the muscle movement but also for other purposes such as, for example, the measuring of the threshold of pain and neurological rehabilitation.

Also advantageously, the contact of the electrode in accordance with the invention with the human body does not cause any particular discomforts to the individual subjected to the therapeutic treatment.

The set forth objects and advantages are better evidenced by the description of the preferred embodiment of the invention given in an indicative manner with reference to the attached illustrations wherein:

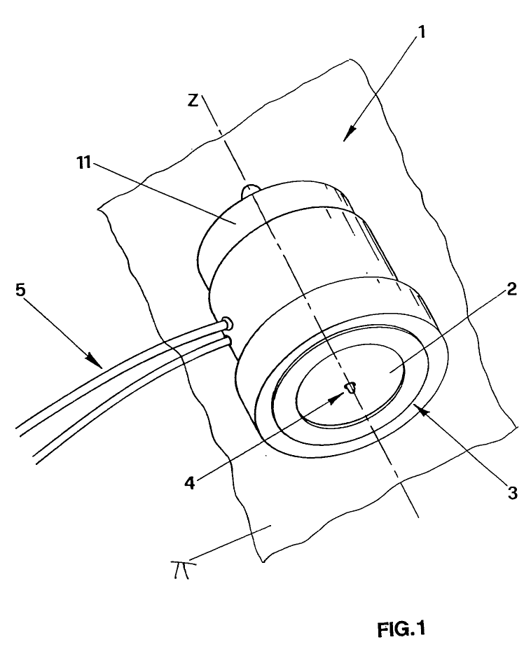

FIG. 1 shows an axonometric view of the electrode in accordance with the invention;

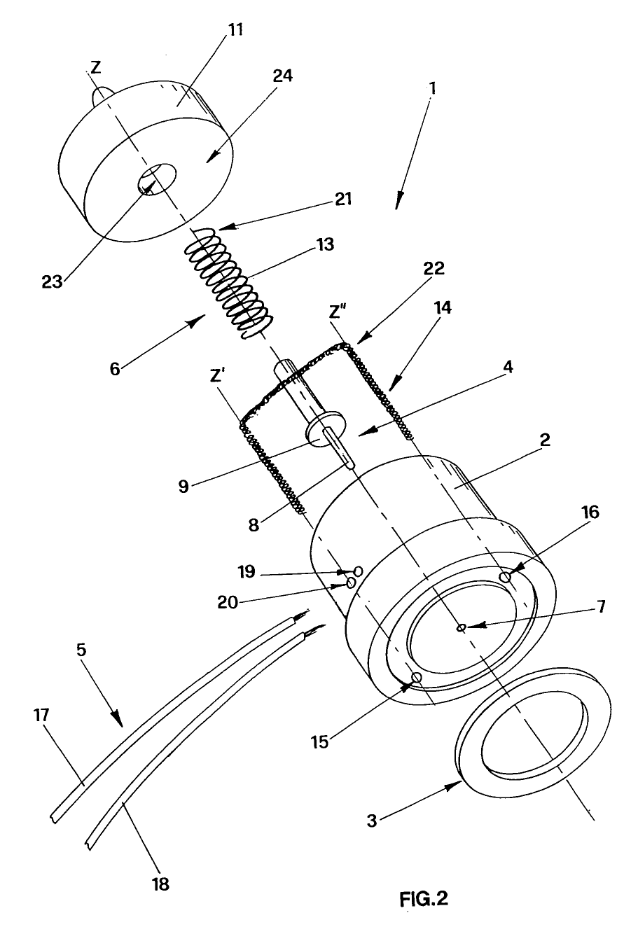

FIG. 2 shows the exploded axonometric view of FIG. 1;

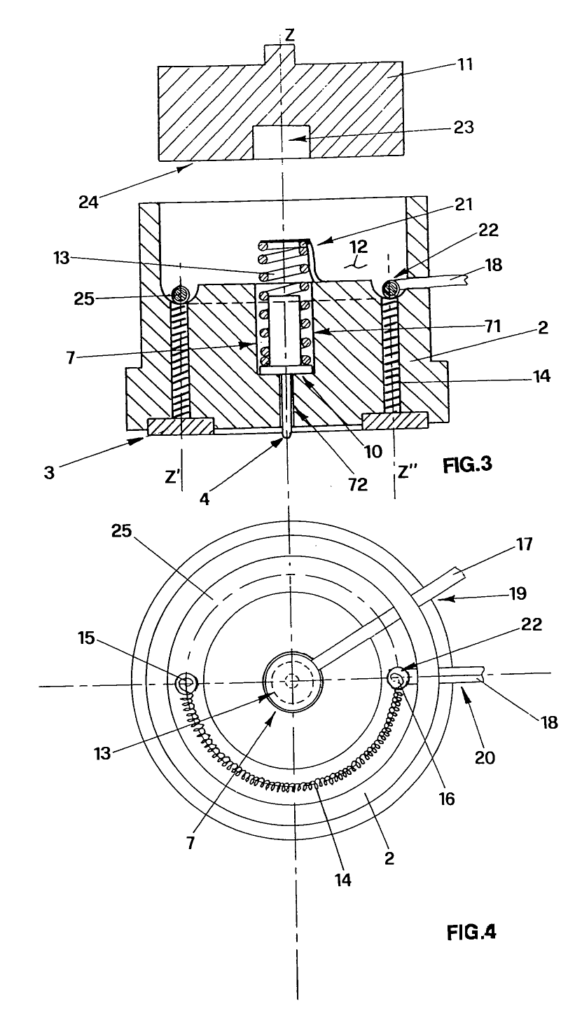

FIG. 3 shows a longitudinal section in accordance with the plane n of FIG. 1 with disassembled cap;

FIG. 4 shows the electrode of FIG. 1 without the cap;

FIG. 5 shows a longitudinal section of a different embodiment of FIG. 1 with disassembled cap;

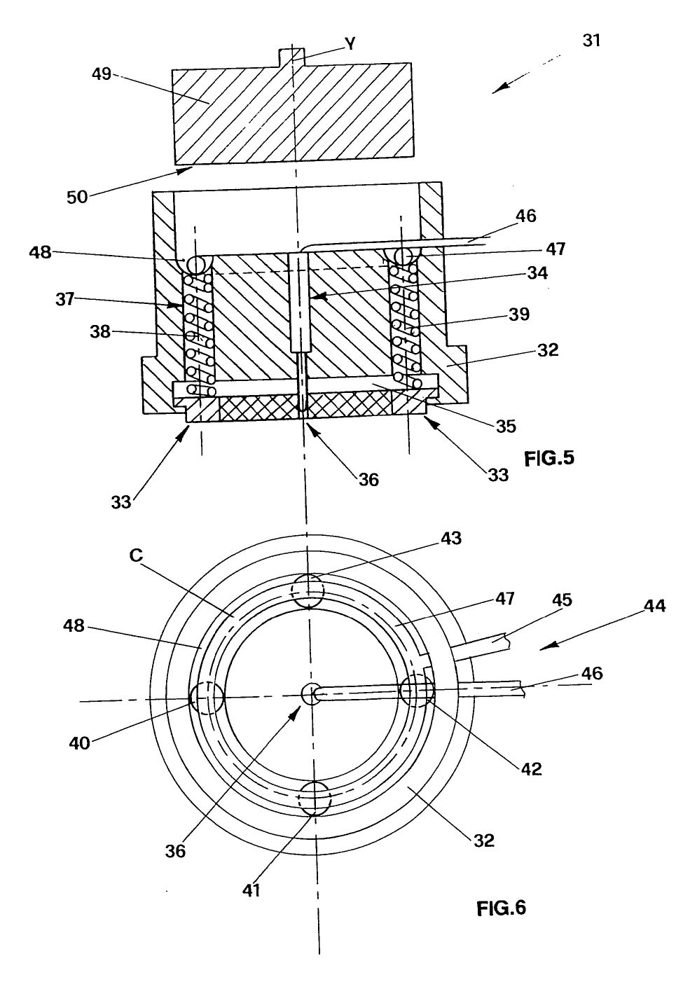

FIG. 6 shows the electrode of FIG. 5 without the cap;

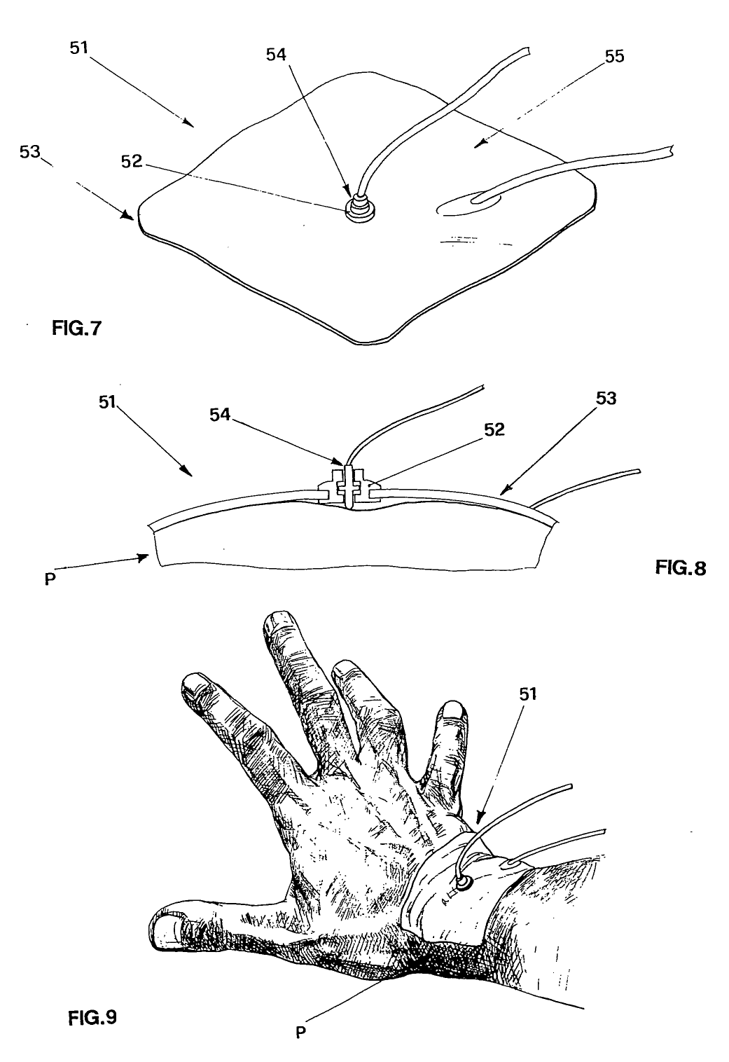

FIG. 7 shows an axonometric view of another embodiment of FIG. 1;

FIGS. 8 and 9 show in accordance with various views the embodiment of FIG. 7 in conditions of application.

The electrode in accordance with the invention is shown in an axonometric view in FIG. 1, wherein it is globally referenced by number 1.

As it can be seen, the electrode 1 comprises an insulating body 2, which in the specific case is of a substantially cylindrical shape and characterized by a symmetry axis Z.

In the insulating body 2 are located a first conductor element, referenced in its entirety by number 3, slightly protruding from the insulating body 2, and a second conductor element, generically referenced by number 4, also protruding from the insulating body 2.

Furthermore, means of electric current conductors are located in the unit 5, utilized to connect the above-mentioned conductor elements 3, 4 to an electro-stimulator, not shown in the next illustration, which generates the electric impulses to be transmitted by means of the two conductor elements 3, 4 to a limited area of the human body to be treated.

In accordance with to the invention, the second conductor element 4 is connected to a flexible structural component, not shown in FIG. 1 but illustrated in FIG. 2 where it is referenced in its entirety by number 6, which ensures the simultaneous contact of both conductor elements 3, 4 with the human body during the therapeutic electro-stimulating treatment.

In more details, and with reference to FIG. 2, the flexible structural component 6 is preferably, but not necessarily, constituted by elastic means.

On its part, the first conductor element 3 is constituted by a metal ring that is affixed to the insulating body 2 on the opposite side towards the human body to be treated.

The second conductor element 4 is constituted by a metal peg inserted into a through hole 7 axially disposed on the insulating body 2.

In accordance with the described preferred embodiment, the second conductor element 4 is movable inside the through hole 7 and of double diameter along the direction of the symmetry axis Z and, when the electrode 1 is not being used, protrudes from the first conductor element 3.

In FIG. 2 can be seen that the metal peg is differently shaped, presenting an extremity 8 that is round with respect to the main body that constitutes it, which extremity is delimited by a concentric ring 9 that protrudes from the metal peg.

As illustrated in a very detailed manner in FIG. 3, the extremity 8 of the metal peg is connected to a first element 72 of lesser diameter than the through hole 7 while the ring 9 is rabbeted against the shoulder 10 that divides the through hole 7 in said first element 72 of lesser diameter and in a second element 71 of greater diameter.

In FIG. 3 can be observed that the electrode 1 comprises a cap 11 set in a housing 12 in the upper portion of the insulating body 2 that acts as baffle wall for the elastic means.

With respect to the elastic means in FIG. 2 and in FIG. 3 it can be noted that, in accordance with the described embodiment, they comprise an helical spring 13 inserted in the second element 71 of greater diameter than the through hole 7.

This helical spring 13 actuates on the second conductor element 4 in order to render it flexible when the electrode 1 is in use.

Another function of the helical spring 13 is that of ensuring the continuity of electric current between the second conductor element 4 and the means of electric current conductors 5, being connected to these as it can be seen clearer as the description continues.

The first conductor element 3 is electrically connected to the means of electric current conductors 5 by means of another conductor 14, shown in FIG. 3, which, in the case of the described embodiment, is in the shape of a helical spring that offers the advantage of being more pliable and of easier orientability.

This conductor 14 is inserted in a couple of through holes 15, 6 having axes of symmetry Z′ and Z′, which run parallel between them and to the axis of symmetry Z of the insulating body 2 and in its center is arranged an annular groove 25 of connection of the two through holes 15, 16, provided in the insulating body 2, in which the conductor 15 is held by applying the cap 11.

The helical spring, that constitutes the conductor 14 is arranged rabetted against the first conductor element 3 that closes the through holes 15, 16 of the opposite side towards the area of the human body to be treated, also in this case to guarantee the continuity of the electric current between the first conductor element 3 and the means of electric current conductors 5.

In accordance with a further embodiment of the invention, not shown in the illustrations that follow, the conductor connected to the first conductor element could be constituted instead of a by helical spring in a much more simple manner by a wire of a conductive material inserted into a through hole and connected on one extremity to the means of electric current conductors and on the other extremity to the first conductor element.

The means of electric current conductors 5 are constituted by a couple of electric wires, numbered 17,18, respectively, that are inserted in corresponding holes 19, 20 provided for on the insulating body 2.

In FIGS. 3 and 4 can be clearly seen that the helical spring 13 presents an extremity 21 connected to the electric wire 17 in order to ensure the passage of the electric current through the second conductor element 4.

Likewise, also the conductor 14 presents the extremity 22 connected to the electric wire 18 in order to allow the passage of the electric current through the first conductor element 3.

The assembly of the electrode 1 is effectuated by affixing the metal ring to the insulating body 2, inserting the metal peg into the through hole 7 and electrically connecting one to the other, by means of the helical spring 13 and the conductor 14, respectively, to the corresponding electric wires 17, 18.

The cap 11 is then applied to the housing 12 on the insulating body 2 which brings about the compression of the mentioned helical spring 13 and of the conductor 14.

While operating, the user places the electrode on the skin of the patient subjected to the therapeutic treatment and brings the second conductor element 4 into contact with the same skin. Continuing with the slight pressure on the electrode 1, the second conductor 4 withdraws towards the inside of the electrode 1 until also the first conductor element 3 does no longer rest on the patient's skin, ensuring in such a manner the passage of the electric charge.

In can be observed that such a functioning can be effectuated because of the fact that the second conductor element 4 is withdrawn from the first helical spring that is wrapped around it.

The contact of the second conductor element 4 with the surface of the human body to be treated pushes, as a matter of fact, the extremity 21 of the first helical spring 13 towards the inside of the dead hole 23 on the inside wall 24 of the cap 11, allowing thus the second conductor element 4 to partially reenter with respect to the fist conductor element 3.

After the point at which the transmission of the electric charge on the human body to be treated is reached, the operator keeps the electrode 1 steady in position, effectuating thus the electro-stimulating treatment.

It is evident that such an embodiment of the invention enables the electrode 1 to adapt itself to the various shapes of the human body without the need of having to use conductive substances of gelatinous type or those on the basis of polymers of conductive type which also reduce the effect and the effectiveness of the electro-stimulating treatment.

It must further be mentioned that the two conductor elements 3, 4 of the electrode, at the moment at which the electric charge is being transmitted to the area of the human body to be treated, must not necessarily be protruding at the same distance from the insulating body 2.

This facilitates the best positioning of the electrode 1 on the area of the patient's body because, as mentioned, the point of contact of the two conductor elements 3, 4 at the same area can hardly be effectuated at the same plane.

FIG. 5 shows a further embodiment of the invention, in which the electrode, globally referenced under number 31, differs from the one described above in view of the fact that now it is the first conductor element, generically referenced under number 33, is yielding with respect to the insulating body 32 and the second conductor element, within the unit referenced under number 34.

As a matter of fact, the first conductor element 33 is movable inside of a round chamber 35 provided for in the insulating body 32 in relation to the side of the electrode 31 turned towards the area of the human body to be treated.

In accordance with the idea of solution of the present invention, the second conductor element 34 is now firm inside of the axial through hole 36 provided for in the insulating body 32.

Therefore, it is evident that in this case, when the electrode 31 is not in use, the first conductor element 33, which moves along the indicated direction of the axis of symmetry Y of the insulating body 32, protrudes from the second conductor element 34.

Another difference of the variant of FIG. 5 with respect to the one described with reference to FIG. 1 concerns the elastic means that constitute the flexible structure, referenced in the unit under number 37, that comprise four helical springs of which only two are visible, referenced under numbers 38 and 39.

As better evidenced in FIG. 6, the four helical springs are inserted in four through holes 40, 41, 42, 43 provided for in the insulating body 32, and arranged in twos symmetrical to the axis of symmetry Y along a circumference C.

The four helical springs are arranged rabetted against the first conductor element 33 in order to render it flexible and to guarantee at the same time the electrical continuity with the means of electric current conductors, globally referenced under number 44.

The above-mentioned means of conduction 44 are constituted, also in this case, by a couple of electrical wires 45, 46 connected on one end to an electrostimulator and on the opposite end, one to the second conductor element 34 and the other one to a wire 47 of conductive material, arranged in the annular groove 48 that connects the four elastic springs to each other.

Lastly, it can be noted that the cap 49, that closes on its top the electrode 31 and that locks the wire 47 in the annular groove 48 does not present a dead hole on its interior wall 50 because the second conductor element 34 is now firm.

When not being used, the first conductor element 33 is compressed in its final position inside of the round chamber 35 of the four helical springs under the pressure exercised by the cap 49.

When in operation, once the operator has placed the electrode 31 on the area of the human body to be treated, the first conductor element 33 moves upwards to the point at which the second conductor element 34 comes into contact with the same area, transmitting in such a manner the electric charge.

Also in this case it is evident that the electrode 31 is suitable to adapt itself to any type of configuration of the human body to be treated because since one of the two conductive poles of the electrode 31 is flexible it follows the contour of the surface with which it enters into contact.

It is also clear that also the electrode 31 allows not having to use further treatment means necessary to ensure the continuity of the electric charge transmission such as, for example, a gelatinous conductive substance inserted between the electrode and the area of the human body to be treated.

The contact between the electrode of the invention and the human body is not painful to the individual subjected to the therapeutic treatment in as much as the conductive poles of the electrode, of which at least one is generally pointed, do not protrude excessively from the insulating body.

FIG. 7 illustrates still a further embodiment of the electrode, herein globally referenced under number 51, which differs from the ones already described by the fact that the flexible structure, referenced under number 55, is constituted by the first conductor element, generically referenced under number 53, separated from the second conductor element, in the assembly referenced under number 54, by means of the interposition of the insulating body 52.

The first conductor element is made out of an intrinsically flexible material, such as, for example, sponge, rubber or the like, that is conductive in order to ensure the passage of the current through it.

FIGS. 8 and 9 illustrate the electrode 51 in condition of application, while effectuating the treatment corresponding to the pulse P of a patient.

It is evident that in accordance with further embodiments of the invention the flexible structure could be carried out by the second conductor element or directly by both.

Based on the foregoing, it is understood that the electrode in accordance with the invention attains all the objects and presents all the aforementioned advantages.

Modifications could be introduced in the constructive phase of the electrode in accordance with the invention, consisting for example in a different shape of the insulting body or a different position of the through holes through which pass the electric wires connected to the electrostimulator.

These holes could be not laterally on the insulating body but above it or on the cap the closes the insulating body itself.

Besides the foregoing, both the first and second conductor elements could be movable.

Further, the conductor elements would not have to necessarily concentric but, by way of example, they could be arranged next to each other.

Also, the helical springs that constitute the flexible means could be connected to the conductor elements in a manner different than above-described, which does not impair the advantage offered by the present patent.

All the described and set forth variants, but not illustrated in the hereto attached tables of designs, are covered by the scope of the inventive concept based on the hereto attached claims, that must be deemed protection of the present patent.

1) An electrode (1; 31; 51) for electrostimulators, comprising an insulating housing (2; 32; 52) in which are located
at least one first conductor element (3; 33; 53) projecting from the insulating housing (2; 32; 52);
at least one second conductor element (4; 34; 54) also projecting form the insulating housing (3; 32; 52);
electrical conductor means (5; 44) appropriate to connect the conductor elements (3, 4; 33, 34; 53, 54) to the electrostimulator,
characterized in that at least one of the conductor elements (3, 4; 33, 34; 53, 54) is connected to a yielding structure (6; 37; 55) suitable to ensure the simultaneous contact of the conductor elements (3, 4; 33, 34; 53, 54) with the human body during the therapeutic treatment.

2) An electrode (1; 31) in accordance with claim 1), characterized in that this yielding structure (6; 37) is constituted by elastic means.

3) An electrode (51) in accordance with claim 1) characterized in that the yielding structure (55) is constituted by at least one of the conductor elements (53, 54).

4) An electrode (1; 31) in accordance with claim 1), characterized in that the first conductor element (3; 33) is constituted by a ring fastened to the insulating housing (2; 32) on its side towards the area of the human body to be treated.

5) An electrode (1; 31; 51) in accordance with claim 1), characterized in that the second conductor element (4) is constituted by a pin inserted in a bolt hole (7) effectuated in the insulating housing (2; 32; 52).

6) An electrode (1) in accordance with claim 5), characterized in that the second conductor element (4) can move freely inside of the bolt hole (7) and that it projects from the first conductor element (3) when the electrode (1) is in a non-use state.

7) An electrode (31) in accordance with claim 1), characterized in that the first conductor element (33) is constituted by a movable ring inside of a round chamber (35) provided for in the insulating housing (32).

8) An electrode (31) in accordance with claim 5), characterized in that this second conductor element (34) is fastened inside of the bolt hole (36) and re-enters as regards the first conductor element (33) when the electrode (31) is in a state of non-use.

9) An electrode (1) in accordance with claim 5), characterized in that the elastic means comprise a helicoidal spring (13) inserted in the bolt hole (7) in order to wind around the second conductor element (4) and render it yielding.

10) An electrode (1) in accordance with claim 1), characterized in that it comprises at least one conductor (14) inserted in a corresponding bolt hole (15, 16) closed by the first conductor element (3) against which it is fitted in order to ensure the electrical continuity with the electrical conductor means (5).

11) An electrode (31) in accordance with claim 2), characterized in that these elastic means comprise at least a couple of helicoidal springs (38, 39) inserted in corresponding bolt holes (40, 41, 42, 43) effectuated in the insulating housing (32) until being fitted against the first conductor element (33) in order to render it yielding.

12) An electrode (1) in accordance with claim 9), characterized in that the helicoid spring (13) presents an extremity (21) connected to the electrical conductor means (5) in order to ensure the continuity of current between this second conductor element (4) and the electrical conductor means (5).

13) An electrode (31) in accordance with claim 11), characterized in that at least one of the helicoidal springs (38, 39), connected between each other by means of a wire (47) of a conductor material, is connected to the electrical conductor means (44) in order to ensure the continuity of current between the first conductor element (33) and the electrical current conductors (44).

14) An electrode (31) in accordance with claim 7), characterized in that this second conductor element (34) is connected to the electrical current conductors (44).

15) An electrode (51) in accordance with claim 3), characterized in that at least one of the conductor elements (53, 54) is constituted by an intrinsically yielding material turned conducive.

16) An electrode (1; 31) in accordance with claim 1), characterized in that the electrical current means (5; 44) are constituted by a couple of electrical wires (17, 18; 45, 46) inserted in corresponding holes (19, 20) effectuated in the insulating housing (2; 32).

17) An electrode (1; 31) in accordance with claim 16), characterized in that the holes (19, 20) are laterally effectuated in the isolating housing (2, 32).

18) An electrode (1; 31) in accordance with claim 1), characterized in that they comprise a cap (11; 49) positioned in a chamber (12) provided for at the top of the isolating housing (2; 32) and that it acts as back wall for the yielding structure (6; 37).

19) An electrode (1; 31) in accordance with claim 5), characterized in that the bolt hole (7) presents a first part (72) of a lesser diameter and a second part (71) of a greater diameter.

20) An electrode (1; 31) in accordance with claim 19), characterized in that the pin presents an end (8) that is connected to the first part (72) with a lesser diameter than the bolt hole (7).

21) An electrode (1; 31) in accordance with claim 20), characterized in that that end (8) of the pin is delimited by a concentric ring (9) protruding from the pin.

22) An electrode (1; 31) in accordance with claim 1), characterized in that the isolating housing (2; 32) presents a substantially cylindrical shape.
